(19) United States
(12) Patent Application Publication
(10) Pub. No.: US 2009/0209738 A1
(43) Pub. Date: Aug. 20, 2009

(54) Method for separating keratinous proteins from materials

(76) Inventors: Robin William Cranston, Victoria (AU); Jacinta Maree Poole, Victoria (AU); Mark Brendan Hickey, Victoria (AU)

Correspondence Address: QUARLES & BRADY LLP, 33 E. MAIN ST, SUITE 900, P.O BOX 2113, MADISON, WI, 53701-2113, US

(21) Appl. No.: 11/665,915

(22) PCT Filed: Oct. 21, 2005
(86) PCT No.: PCT/AU05/01637

§ 371 Date: May 1, 2009

(30) Foreign Application Priority Data
Oct. 21, 2004 (AU) 2004906135

(51) Int. Cl. C07K 1/14

(52) U.S. Cl. 530/422

(57) ABSTRACT

A process for separating keratinous proteins from a keratin-containing material, the process comprising the steps of: subjecting the keratin-containing material to reduction in a liquid medium to solubilise the keratinous proteins under conditions that minimise hydrolysis of the keratinous proteins, to yield a solution of keratinous proteins and undissolved solids; subjecting the solution of keratinous proteins to peroxide oxidation, without any intervening keratin precipitation step; and separating the solution of keratinous proteins from the undissolved solids prior to, at the same time as, or following the oxidation step. Preferred conditions for performing the reduction step involve contacting the keratin-containing material with a solution of an alkali metal sulfide reducing agent at a temperature of between 25 C and 50 C for a time of between 30 and 90 minutes, assuming atmospheric pressure. The peroxide oxidation is suitably carried out within not more than 4 hours after the reduction step, and involves reducing the pH of the solution to a level not less than pH 10, although pH 11.3 is most preferred. The product is demonstrated to have a principal fraction that has a molecular weight above 10 kDa, reflecting that the disulfide bonds in the keratinous proteins are broken without hydrolysis of the proteins.

The present invention relates to a method and arrangement for separating keratinous proteins from a keratinous material, and a keratin protein product obtained by the method.

Many techniques disclosed in the art for extracting keratinous proteins from keratin-containing materials involve treatment of the keratin-containing materials under extreme conditions that cause hydrolysis of the proteins. Consequently, the molecular weight of the keratinous proteins yielded from such processes is lower than that of the original proteins. It is reported in Harrap and Woods (Biochem J. 902, 1964, 16) that monomeric proteins from feather have a molecular weight of 10.4 kDa. Consequently, the keratinous proteins yielded from the processes of the prior art generally have a molecular weight of less than 10.4 kDa.

It has been postulated by the present inventors that for many potential applications, it would be desirable for the proteins to be prepared in a manner that minimises reduction of or preserves the naturally occurring molecular weight. This is especially the case if the intended application is to provide an alternative to a polymeric material when high molecular weights are desirable. Maximising the molecular weight of extracted proteins would also maximise the chemical possibilities for later protein modification.

Many of the processes disclosed in the art for preparing keratinous proteins from a keratin-containing material are not appropriate for scale-up to an industrial process. For instance, some methods disclosed in the prior art involve physical separation with “cheese cloth”. Some processes evolve sulfur dioxide or hydrogen sulfide gas, which, although not a prohibitive problem on a laboratory scale, does cause significant problems when scaled up. Moreover, some processes involve the formation of a gel, which is very difficult to handle in an industrial scale process.

Accordingly, there exists a need for a method for separating keratinous proteins from a keratinous material, in a form that provides opportunities for further chemical or physical modification, and utilising a method which is practicable for industrial scale processing.

### SUMMARY OF INVENTION

According to a first aspect there is provided a process for separating keratinous proteins from a keratin-containing material, the process comprising the steps of:
i. subjecting the keratin-containing material to reduction in a liquid medium to solubilise the keratinous proteins under conditions that minimise hydrolysis of the keratinous proteins, to yield a solution of keratinous proteins and undissolved solids;
ii. subjecting the solution of keratinous proteins to peroxide oxidation, without any intervening keratin precipitation step; and
iii. separating the solution of keratinous proteins from the undissolved solids prior to, at the same time as, or following the oxidation step.

The process outlined above eliminates the risk of hydrogen sulfide or sulfur dioxide being produced. The production of these gases in an industrial process is highly problematic. Various embodiments of the invention provide further advantages over the prior art.

Preferably, the reduction is performed to a level where the disulfide bonds of cystine present in keratinous proteins are broken to form cysteine residues, but without hydrolysis. This can be visually assessed by detecting a principal molecular weight band corresponding to a molecular weight that is above 10 kDa on sodium dodecylsufate—polyacrylamide gel electrophoresis (“SDS-PAGE”), compared to a known standard. This band reflects that a majority of the keratinous proteins solubilised in step i. have a molecular weight of 10.4 kDa or above. Generally such electrophoresis results reflect that at least 90% of the keratinous proteins solubilised in step i. have a molecular weight in this range. In the case of wool derived keratins, if the reduction effects reduction of the disulfide bonds in the cystine residues to cysteine, without hydrolysis of the keratinous proteins, this corresponds to at least 90W, and typically at least 94% of the keratins solubilised in step i. having a molecular weight greater than 11 kDa.

According to a second aspect, there is also provided a product produced by the process set out above.

According to a third aspect, there is provided an assembly for producing keratinous proteins from a keratin-containing material, the assembly comprising:
i. washing apparatus for washing a keratin-containing raw material;
ii. a digestion vessel for reducing the keratin-containing material to produce a solution of keratinous proteins and undissolved solids;
iii. an oxidation treatment zone for oxidising the solution of keratinous proteins;
iv. separating apparatus for separating the undissolved solids from the solution of keratinous proteins;
v. ultrafiltration apparatus for removal of excess salts and concentration of the solution of keratinous proteins; and
vi. conveyors for conveying material or streams containing the keratinous proteins from the washing apparatus to the ultrafiltration apparatus via each other component of the assembly.

Preferably, the assembly further comprises:
vii. a mill for fragmenting the keratin-containing raw material prior to reduction.

This mill is suitably located prior to the digester in the assembly.

The oxidation treatment zone may be constituted by a vessel, a conveyor, the digestion vessel, or any other apparatus component or region in which the solution of keratinous proteins can be contacted with peroxide oxidising agent. The separating apparatus is located following the digestion vessel in the assembly (in which the solution of keratinous proteins and undissolved solids is produced), but may be located before or following, or may be combined with the oxidation treatment zone.

### BRIEF DESCRIPTION OF THE FIGURES

FIG. 1 is a schematic diagram illustrating the process stages for the separation of keratinous proteins from a keratin-containing raw material, according to one preferred embodiment of the invention.

FIG. 2 is a photograph showing a Tris-Tricine gel electrophoresis of keratinous protein samples obtained by the process, the lanes from left to right representing precision MW standard; oxidised feather@30° C.; oxidised feather@45° C.; reduced feather@30° C.; and the polypeptide standard.

FIG. 3 is a photograph showing a Tris-HCl 15% gel electrophoresis of keratinous protein samples obtained by the process, the lanes from left to right (with lanes 1 and 3 empty) being: Lane 2—Std, Lane 4—pH 11.3 (1:5), Lane 5—pH 11.3 (1:10), Lane 6—pH 10.8 (1:5), Lane 7 pH 10.8 (1:10), Lane 8—pH 10.1 (1:5), Lane 9—pH (1:10) Lane 10—Standard.

### DETAILED DESCRIPTION OF THE INVENTION

The preferred embodiments of the invention will now be described in further detail.

### Keratin-Containing Material

The keratin-containing material may be derived from any keratinous source, including feather, wool, hair, animal hoof or claw, animal horn, animal scale, or any other keratinous epidermal material, or a mixture of the above. One preferred source of the keratin-containing material is feather, such as chicken and/or turkey feather. Another preferred source is wool. The keratin-containing material fed to step (i) of the process may have been subjected to washing and optionally a fragmentation process. Such raw material preparation stages are described in further detail below.

### Raw Material Preparation

The keratinous raw material is advantageously washed and optionally fragmented into smaller particles to be in a suitable form for subjecting to the reduction step of the process. In the case of non-feather materials, the preparation advantageously includes scouring and washing to remove extraneous materials, greases and lipids. In such materials, such as wool, fragmentation may not generally be necessary, although there may be some practical or economical advantages for using short wool.

According to one suitable embodiment of the invention, the keratinous material is washed with warm water containing a suitable surfactant and rinsed with water. The keratinous material may be dried if it is not intended to be processed immediately after the washing stage.

In the case of feathers, these are advantageously washed and fragmented into smaller fragments to aid digestion in the reduction step of the process. The washing may be conducted by washing in surfactant-containing water, as described above. Thereafter, according to one embodiment of the invention, the washed feathers are fragmented by mincing and/or milling. Similarly, dry feathers may also be fragmented by mincing or milling prior to digestion in the reduction stage. Fragmenting may be effected in any suitable apparatus, such as a mincing machine of a type used by butchers, or a rotary blade (e.g. Wiley) mill. Fragmentation enables a greater amount of keratin to be extracted from the feather material, but at the expense of the energy and equipment costs for performing the fragmentation. In most situations the fragmentation step would be warranted.

### The Reduction Step

The keratin-containing material, which may according to one embodiment be a feather or wool material, is subjected to reduction in a liquid medium to solubilise the keratinous proteins. This stage of the process is sometimes referred to as “digestion”.

The reduction step effects reduction of the disulfide bonds in the cystine residues of the keratins in the keratin-containing material to cysteine (containing terminal thiol groups) to break the linked keratin protein strands into smaller strands. In the case of feather keratins, the smaller strand length is around 10.4 kDa, although some larger strands may remain, but preferably the majority of the product is the 10.4 kDa product (i.e. just above 10.0 kDa). In the case of wool keratins, there are a variety of proteins of differing molecular weights between the disulfide bonds. A great majority, above about 94% of these, are greater than 11 kDa. About 6% of the proteins, known as the “high Gly/Tyr” proteins, have a molecular weight of between 9-13 kDa, and thus a very minor percentage (less than 6%) of the reduced proteins will include some protein of between 9-11 kDa in molecular weight. Visual assessment of gel electrophoresis results will however reflect that the principal molecular weight fraction is above the 10 kDa level.

The reduction stage is suitably conducted under conditions that minimise the hydrolysis of the keratinous proteins. In other words, the reduction conditions effect breaking of the disulfide bonds, but are not so severe as to cause hydrolysis of the keratinous proteins. The extent of hydrolysis of the keratinous proteins can be measured by reference to the molecular weight of the keratinous proteins following the reduction stage. If the molecular weight of the protein product is below the levels indicated above for the particular keratin types, then the reduction conditions have not been such as to minimise hydrolysis of the keratinous proteins. In particular, if the majority of the keratinous proteins have a molecular weight below 10.4 kDa for feather, or a majority of the keratinous proteins are below 10.4 kDa (or more specifically 11 kDa) for wool (noting that up to 6% of the wool proteins will be in the 9-13 kDa range when the conditions are as required), the reduction conditions are too harsh. Under typical conditions of the present process, visual assessment of the type described in the Examples will reveal that the principal molecular weight fraction of the product corresponds to a product having a molecular weight of 10.4 kDa or higher. This visual assessment corresponds to the product having at least 90%, usually at least 95% of the product in this molecular weight range. Due to contaminating material from harvested feathers, such as fragments or adhering flesh tissue and blood in the quill, the product may not be 100% free from lower molecular weight material.

The protein size is generally assessed qualitatively using polyacrylamide gel electrophoresis (PAGE) of the extracted feather protein, with a lower molecular weight cut-off of 5 kDa. It is noted that all references to assessment of the molecular weights are based on polyacrylamide gel electrophoresis, compared to a standard with known molecular weight fractions for comparison, and with a lower molecular weight cut-off of 5 kDa. In a qualitative PAGE run on a feather keratin product, there is observed a heavy band corresponding to 10.4 kDa, and light bands at around 25 kDa, 35 kDa and 50 kDa. No significant bands can be detected below the 10.4 kDa band, down to the 5 kDa cut-off. For a wool keratin product, the bands will generally reflect the constituent keratinous protein fractions of wool that are able to be solubilised, which are as follows:

| Soluble Protein | Low | High | Ultrahigh | High |
|---|---|---|---|---|
| Fraction | sulfur | sulfur | sulfur | Gly/Tyr |
| Amount (%) | 58% | 18% | 8% | 6% |
| Sulfur Content (%) | 1.5-2.0 | 4-6 | 8 | 0.5-2.0 |
| Mol Mass (kDa) | 45-50 | 14-28 | 28 | 9-13 |

Reference: Wool Science-The Chemical Reactivity of the Wool Fibre. John A Maclaren and Brian Milligan, Science Press (1981) page 13.

To minimise hydrolysis of the keratinous proteins, an appropriate balance between reduction time and temperature must be achieved. The following figures apply for reductions carried out at atmospheric pressure, and modifications to the times and temperatures are to be made accordingly if conducted under pressure. Generally, in the case of feather keratins, the reduction time should be 1 hour or less for reaction temperatures of 50° C. or less, and for example is suitably 45° C. for a digestion lasting about 45 minutes at atmospheric pressure. However, should the reduction temperature be at 25° C. or less, the reduction stage may be conducted over a period of up to 1.5 hours or more. In the case of wool keratins, the reduction time may be from 1 hour to 72 hours at 25° C., with lower temperatures enabling the longer reduction times, and higher temperatures enabling shorter reduction times. For temperatures between 25° C. and 50° C., the time period is suitably within the range of 30 minutes to 1.5 hours, assuming atmospheric pressure.

The reduction stage may be conducted in an air or oxygen-containing atmosphere, or may alternatively be conducted in an oxygen-reduced or oxygen-free atmosphere. The reduction may be conducted in an inert gas atmosphere, for instance in a nitrogen gas atmosphere. It was postulated that performing the reduction under nitrogen atmosphere could further diminish the tendency of the keratinous protein solution to form a gel. Subsequent testing indicated that the gelling properties were not so different as to be noticeable.

The reducing agent may be any suitable reducing agent that effects digestion of the keratinous proteins, whilst minimising hydrolysis thereof. According to a preferred embodiment of the invention, the reducing agent is an alkali metal sulfide, such as sodium or potassium sulfide. In the case of alkali metal sulfide reducing agents, these are suitably used in an amount of from 5 grams per 100 litres to 1000 grams per 100 litres, depending on the nature of the keratin containing material (e.g. feather or wool), the temperature, pressure, pH and time conditions. An appropriate balance can be determined by reasonable trial and error, using the molecular weight information from the reduced product as a guide, in conjunction with the examples presented herein. In general, the conditions will be more severe for wool-based keratinous materials.

Preferably, the reduction stage is conducted in the presence of an alkali, and more preferably an alkali metal hydroxide. Suitable examples of alkalis are sodium and potassium hydroxide.

It is also possible for further additives to be used in the reduction stage. Suitable additives include surfactants and so forth. The addition of surfactants including anionic, cationic and non-ionic surfactants in various stages during production in some cases may be used to improve the characteristics of the final keratinous protein product.

According to one embodiment, the alkali is added in an amount sufficient to adjust the pH to 12.0-13.5. According to one preferred embodiment, the pH is adjusted to 13.0.

Reduction of the keratinous proteins will continue to occur after the material is discharged from the reduction vessel, unless the reduction is ended in the reduction vessel by adding a suitable reagent. Accordingly, it has been found that the subsequent stages need to be controlled to ensure that the continued reduction does not proceed to the extent that hydrolysis occurs.

### Separation of Undissolved Solids

The keratinous proteins are separated from the undissolved solids using techniques that avoid excessive gel formation, via aggregation of the keratinous proteins.

With small scale processing, the separation of the solubilised protein from undissolved solids can be performed by centrifugation followed by rapid vacuum filtration to remove the remaining fine particulate matter. However, according to a preferred embodiment particularly suited to larger scale processing, vacuum or pressure filtration apparatus is used. The most suited apparatus tested is a filter press, although other filters that continuously or semi-continuously present a fresh filtration surface to the feed material being subjected to separation are anticipated to be feasible. It is noted that rotary drum filters are not as suited to the process as the filter press. The preferred type of filter press is a membrane filter press—either of the mixed plate or full plate type. A large range of filters in this class are available commercially and are described for example in the Filters and Filtration Handbook, Third Edition (1994) Christopher Dickenson, Elsevier Advanced Technology.

If a continuous decanting centrifuge is used, it is desirable for the G forces to be sufficiently high to achieve a high degree of separation of solids from the protein solution, to avoid the need for further separation stages to remove residual material.

According to a preferred embodiment, the separation step is conducted prior to aggregation of the stabilised keratinous proteins. Preferably, the solid separation is conducted within a period not greater than 4 hours after the reduction step.

According to one particular embodiment of the invention, the separation step is commenced within a period of 1 hour after the completion of the reduction step, and the undissolved solids separated are subjected to a second stage of reduction. This second stage of reduction, like the first stage, is conducted in a liquid medium to solubilise the keratinous proteins therein under conditions that minimise hydrolysis of the keratinous proteins, to yield a solution of keratinous proteins of similar qualities to those yielded in the first stage. The solids remaining from the second stage of reduction can be considered to be waste undissolved solids.

The secondary stage of reduction/digestion may be conducted under the same conditions as the first or primary stage, or under modified conditions, to recover further keratinous protein for further processing in the process. The “secondary solution” of keratinous proteins may be combined with the “primary solution”, or may be kept separate. One reason for keeping the secondary solution of keratinous proteins separate from the primary solution is due to the fact that the amount of protein and the molecular weight of the extracted protein from the secondary reduction/digestion may be variable (and generally lower than the primary stage). During alkaline digestion of keratins, especially in the presence of sulfides, there is progressive conversion of disulfide bonds to lanthionine and lysinoalanine residues that are not then able to be cleaved by additional reducing reagent, such as sulfide.

Preferably, if conducted, the secondary reduction/digestion is conducted within a period not greater than four hours following completion of the primary reduction stage of step i. If the solids are left in alkaline conditions for greater than four hours before further digestion, the amounts of lanthionine and lysinoalanine residues have a significant effect on the secondary yields.

### Peroxide Oxidation

The peroxide oxidation step may be conducted with any suitable peroxide oxidising agent, such as those selected from the group consisting of hydrogen peroxide, sodium peroxide, peroxy acids, sodium perborate and sodium percarbonate. For economic reasons, hydrogen peroxide is preferred. The hydrogen peroxide is typically supplied in solution form. The concentration of the aqueous solution may vary, but is suitably between 10% to 50%, such as about 30%.

The peroxide oxidation step effectively ends the reduction of the keratinous proteins in the keratin-containing material. The peroxide reacts with or quenches the reducing agent remaining in the solution. Accordingly, the time between commencement of the (primary) reduction stage and commencement of the oxidation stage is preferably less than 6 hours.

The peroxide oxidation step effects oxidation of the thiol groups of the cysteine residues to cysteic acid. Accordingly, the degree of oxidation is preferably sufficient to effect complete conversion to cysteic acid.

Preferably, the peroxide is used in an amount to reduce the pH to a level not below 10.0, preferably not below 10.5, more preferably not below 10.8. According to amino acid analysis results performed, it is preferred that the pH be reduced with peroxide to approximately 11.3.

According to one embodiment of the invention, the pH of the solution of keratinous proteins is not reduced below pH 10, preferably 11, from the commencement of the reduction step and prior to completion of the peroxide oxidation step.

According to a preferred embodiment of the invention, the keratinous proteins in the solution are not precipitated prior to conducting the peroxide oxidation step.

### Solids Treatment

While the keratinous proteins extracted from the original keratin-containing material are still in solution, the remaining undissolved solids (“waste solids”) are separated. This separation can occur prior to, at the same time as, or following the oxidation step. In one suitable embodiment, the solids are separated after reduction and prior to oxidation.

These “undissolved solids” can be subjected to the secondary reduction described above, or sent to waste.

In the situation where the solution of keratinous proteins is separated from the undissolved (waste) solids prior to peroxide oxidation, preferably there are no other intervening treatment steps between the separation and the peroxide oxidation. In addition, peroxide oxidation of the solution of keratinous proteins preferably commences within a period of not more than 1 hour after completion of the separation step.

For the avoidance of any confusion, it is noted that the “solids” referred to here are not precipitated keratins, but are the solids remaining after extraction of the keratins therefrom. Although these waste solids may contain some residual keratinous proteins, they may conveniently be referred to as “non-keratinous solids”, since the keratins to be subjected to further processing have been removed therefrom.

The waste solids may be treated to eliminate remaining sulfide, neutralised, and supplied as an additive for feedstock or fertiliser manufacture. Particularly according to the embodiment involving extraction of keratinous proteins from feathers, the reuse of the solids in such applications minimises environmental waste. According to this embodiment, only the scour washings and salts following oxidation of the digest liquors may need to be discharged to effluent treatment.

### Optional Neutralisation

According to one embodiment of the invention, at the conclusion of the peroxide oxidation stage, the pH of the protein solution is adjusted to a neutral pH or between pH 7-10 to avoid cleavage of peptide bonds by alkaline hydrolysis. The pH of the protein solution may be neutralised or adjusted by any suitable acid. This includes mineral acids such as hydrochloric, sulfuric, carbonic, nitric and boric acids or organic acids such as formic, acetic and glycollic acid.

Such a neutralised protein solution may be used, without further processing, in some applications that are not affected by salts remaining in the solution. The neutralised protein solution may be used in solution form, or may optionally be dried. The dried protein is readily soluble in water in contrast to unoxidised protein.

The potential techniques for drying the salt-containing neutralised protein solution are the same as those described below in the context of the embodiment involving desalted and concentrated keratinous proteins.

Consequently, according to a preferred embodiment of the invention, the process comprises the further step of:
iv. neutralising the oxidised solution of keratinous proteins.

### Desalting

As explained above, the salt-containing neutralised protein solution may be used in some applications without further processing. However, according to a preferred embodiment of the invention, the process comprises the further step of:
v. desalting the neutralised solution of keratinous proteins.

Desalting refers to the removal of the salts generated during the neutralisation stage. The salts present would depend on the alkali used in the neutralisation step.

One particularly suitable technique for desalting the keratinous protein solution is desalting by diafiltration using ultrafiltration technology. Diafiltration is effective in separating proteins of around 1 kD-1000 kDa from small peptides and salts. The diafiltration is conducted using a cross-flow ultrafiltration membrane. Preferably, three or more volume exchanges of water (pH 7) or buffer solution are used to remove excess salts. However, five or more volume exchanges of water or buffer yield higher purity product. Currently, about 10 volume exchanges are being used.

During diafiltration pure water or a weak buffer solution in water is introduced to replace the salts in the starting solution.

Diafiltration of the oxidised protein produced according to the process of the present invention has been found to proceed with excellent yields, and with minimal blocking of the filtration membrane.

### Concentration

The desalted keratinous protein solution is optionally subjected to concentration.

Concentration may be effected by any suitable method. Ultrafiltration is one such method. Specifically, ultrafiltration apparatus may be used to remove or separate some water from the keratinous protein solution to thereby concentrate the solution.

### Ultrafiltration Technology

Larger scale ultrafiltration apparatus is available, and is in use in the dairy industry. Trials have been conducted to determine the capability of ultrafiltration to effectively separate keratinous proteins of 10.4 kDa and above from salts, and concentrate the solution, without blocking of the membrane or aggregation of the protein. This is achieved due to the control of the process steps as outlined above, in which functional groups which in the past have caused aggregation or precipitation of the protein are converted into stable functional groups.

Membrane cartridges or cassettes are available from various suppliers such as Millipore which are capable of effecting the required separation and concentration. The cut-off molecular weight of these membranes are typically somewhat below the 10.4 kDa level of the reduced and oxidised keratins. Usual cut-off values for membranes used in the present applications are less than 10 kDa but more than 5 kDa.

The diafiltration and concentration in the ultrafiltration stage can be conducted continously or semi-continously, or in batches. Semi-continous processing is particularly appropriate, to enable multiple passing of a quantity of solution through the apparatus prior to treatment of the next quantity.

### Optional Keratinous Protein Modifications

Following production of a desalted, and optionally concentrated, keratinous protein solution, it is possible to conduct side protein chain additions or modifications, as required for any particular application. As a consequence, according to one embodiment of the invention, the process comprises the further step of:
vi. modifying the keratinous protein to produce a modified keratin-based product.

By way of example, the keratinous protein can be chemically modified by the introduction of carboxyl, amide, hydroxyl, aryl, alkyl or aromatic groups, either separately or in combination. Depending on the type of modification made, the modified keratin-based products may remain soluble, or substantially soluble in water.

### Optional Drying

According to one embodiment of the invention, the process comprises the further step of recovering the keratinous proteins in solid form from the solution, for example by drying the solution of oxidised keratinous proteins. This drying step is suitably conducted following desalting and/or concentration of the keratinous protein solution.

Suitable techniques for drying the protein include freezing and spray drying. Spray drying is a preferred method due to economic reasons. Nevertheless, large scale freeze drying equipment is commercially available, and can process around 1,000 litres of solution in 24-36 hours. Due to the capital and running costs, freeze drying typically would be used only for high-value added applications.

### EXAMPLES

### Example 1

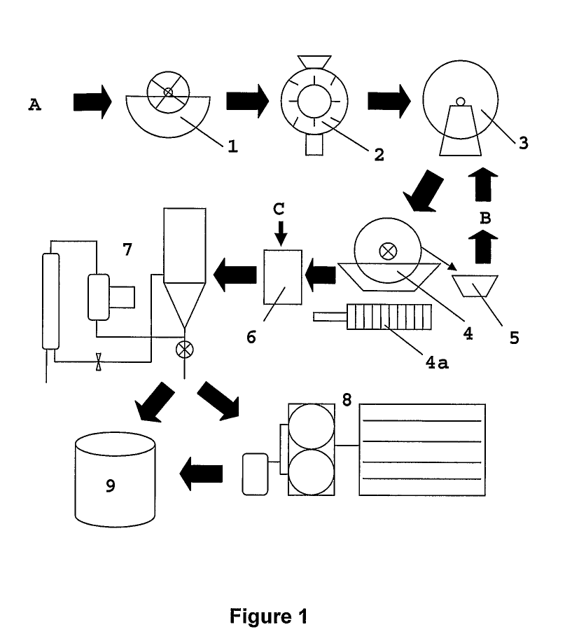

FIG. 1 illustrates schematically a process of one embodiment of the invention.

The embodiment illustrated in FIG. 1 involves extraction of keratinous proteins from feathers A. The feathers A, are washed and scoured in washing apparatus (1) to remove blood, dirt and other contaminants. As a washing apparatus, a stainless steel tumbling vessel of 250 L capacity manufactured by Dose GmbH was used. The washed feathers are then fragmented, or milled, in a mill (2). The mill used was a butcher's mincing machine supplied by Butcher's Suppliers Pty Ltd of Australia.

The milled feather product is then transferred into a digestion vessel (3) in which the milled feathers are reduced to produce a product containing a solution of keratinous proteins, and undissolved solids. The digestion vessel is another Dose 250 L stainless steel tumbler. The product mixture is transferred to a 0.87 m² rotary drum vacuum filter (4) supplied by Chemical Plant and Engineering Pty Ltd for separating the solids from the solution of keratinous proteins. This apparatus has since been replaced in the assembly with a filter press (4a). The filter press is a membrane filter press such as supplied by Diemme, Italy. The solids are collected in vessel (5), and subjected to a secondary stage of reduction/digestion. In FIG. 1, the arrows indicate that the solids are returned to digestion vessel (3), however in general the solids returned to secondary digestion/reduction are processed separately.

Following the solids filtration stage in the rotary drum vacuum filtration apparatus (4) (now replaced by the filter press 4a), the liquid stream discharged from the filter is subjected to oxidation in an oxidation vessel (6). The oxidation vessel was a 250 L HDPE stirred tank.

According to the process illustrated in FIG. 1, neutralisation of the product of the oxidation stage is conducted in the oxidation vessel (6). However, variations of this are possible, and the neutralisation may be conducted in a separate vessel or region of the processing plant.

Following this stage, the (neutralised) oxidised keratinous protein solution is subjected to diafiltration and concentration in the ultrafiltration apparatus (7). The ultrafiltration apparatus used had an array of Millipore™ cellulose filter cartridges with a nominal molecular weight cut off (MWCO) of 5 kDa.

As represented by the arrow pointing left in FIG. 1, the concentrated keratinous protein solution may be the product (9) of the extraction process. Alternatively, as represented by the arrow pointing right in FIG. 1, the concentrated keratinous protein solution may be subjected to drying in spray drying apparatus (8).

The process may be conducted as a continuous process or as a batch process. However, the continuous process is preferred. The term “continuous” encompasses semi-continuous.

Although not directly illustrated in FIG. 1, the product of the secondary stage of digestion/reduction is subjected to solids filtration in appropriate apparatus, such as rotary drum vacuum filtration apparatus or preferably a filter press. The waste solids separated in this apparatus are sent to waste processing and used in the production of feedstock or fertiliser.

### Example 2

The following process was conducted in the apparatus illustrated in FIG. 1 and described above.

10 kg of feathers were washed and scoured in the washing apparatus using water and surfactant Baymol A™ from Bayer. The washed feathers were wet milled in the mill, and the minced product transferred to the rotary drum mixer. In the rotary drum mixer, the milled feathers were digested with a solution of 320 grams of sodium sulfide (calculated as 100% Na₂S) in 100 litres of water at 25° C. (concentration therefore 3.2 g/l). The pH of the reduction mixture was adjusted to pH 13 with sodium hydroxide.

The milled feathers were subjected to digestion over a period of 45 minutes. Following digestion, the product was immediately transferred to the filtration apparatus in which the solids were separated from the solution containing keratinous proteins. The separation stage was conducted over a period of 1 hour.

The solution of keratinous proteins is transferred to an oxidation vessel. In this vessel 30% hydrogen peroxide was added to the solution until the pH had dropped to 11.3.

The oxidised solution was then subjected to diafiltration by using tangential flow filtration against several volume exchanges of water (pH 7) to desalt the solution and reduce the pH to about 10. The same ultrafiltration apparatus was used to concentrate the desalted protein solution to yield a 10% protein solution. The protein yield was 40%.

### Example 3

The following process was conducted in the apparatus illustrated in FIG. 1 and described above, with the exception that the wool was not fragmented in a mill following washing.

In a rotary drum mixer, 1 kg of scoured wool was digested with a solution of 100 grams of sodium sulfide (calculated as 100% Na₂S) in 20 litres of water at 25° C. (concentration therefore 5 g/l). The pH of the reduction mixture was adjusted to pH 13 with sodium hydroxide.

The wool was subjected to digestion over a period of 24 hours. Following digestion, the product was immediately transferred to the filtration apparatus in which solids were separated from the solution containing keratinous proteins. The separation stage was conducted over a period of up to 60 minutes.

The solution of keratinous proteins is transferred to an oxidation vessel. In this vessel 30% hydrogen peroxide was added to the solution until the pH had dropped to 11.3. No precipitation occurred before the oxidation, as the pH was maintained above 10 (in fact, above 11) from the commencement of the reduction step, to the completion of the peroxide oxidation step.

The oxidised solution was then subjected to diafiltration by using tangential flow filtration against several volume changes of water (pH 7) to desalt the solutions and reduce the pH to about 10. The same ultrafiltration apparatus was used to concentrate the desalted protein solution to yield 5% protein solution.

Various modifications may be made to this arrangement. In particular, the oxidation vessel (6) can be removed, and the oxidation conducted in the same vessel as the reduction (3). This would result in further consequential variations in the arrangement of the apparatus.

### Example 4

The following process was conducted in the apparatus illustrated in FIG. 1 and described above.

1.5 kg of feathers were washed and scoured in the washing apparatus using water and surfactant Baymol A™ from Bayer. The washed feathers were wet milled in the mill, and the minced product transferred to the rotary drum mixer. In the rotary drum mixer, the milled feathers were digested with a solution of 65 grams of sodium sulfide (calculated as 100% Na₂S) in 15 litres of water at 45° C. (concentration therefore 4.3 g/l). The pH of the reduction mixture was adjusted to pH 13 with sodium hydroxide.

The milled feathers were subjected to digestion over a period of 45 minutes. Following digestion, the product was immediately transferred to the filtration apparatus in which the solids were separated from the solution containing keratinous proteins. The separation stage was conducted over a period of 1 hour.

The solution of keratinous proteins is transferred to an oxidation vessel. In this vessel 30% hydrogen peroxide was added to the solution until the pH had dropped to 11.3.

The oxidised solution was then subjected to diafiltration by using tangential flow filtration against several volume exchanges of water (pH 7) to desalt the solution and reduce the pH to about 10. The same ultrafiltration apparatus was used to concentrate the desalted protein solution to yield a 17% protein solution. The protein yield was 31%.

### Example 5

Trials were conducted to optimise the conditions for the extraction of keratin from keratin-containing materials, with a focus on the temperature of reduction, and specific pH conditions for the oxidation step. Examples 5a and 5b were preferred on feather, and 5c and 5d on wool. The conditions were as set out in the attached tables, noting that any conditions or aspects of the examples not mentioned are as described in Example 4.

### Example 5a

| Feather (milled) | 500 | g | 45 minutes @ 30° C. |
|---|---|---|---|
| Water | 5 | L | |
| Na₂S (as 100% Na₂S) | 16 | g | |
| NaOH (50%) | 50 | ml | |
| Centrifuge | | | 7000 rpm for 10 minutes |
| Solids recovered | 1977.6 g @ | | Total digested 45% |
| | 14.03% solids | | |
| Vacuum filter | | | Recover 2800 ml protein |
| Oxidise protein | To pH 11.3 | | |
| H₂O₂ (10%) | 285 | ml | |
| Diafilter (with 1 mM EDTA) | 28 | L | |
| Concentrate protein | 1500 | ml | |
| Protein concentration | 5.37% | | |

### Example 5b

| Feather (milled) | 500 | g | 45 minutes ® 45° C. |
|---|---|---|---|
| Water | 5 | L | |
| Na₂S (as 100% Na₂S) | 16 | g | |
| NaOH (50%) | 50 | ml | |
| Centrifuge | | | 7000 rpm for 10 minutes |
| Solids recovered | 1426 g @ | | Total digested 59% |
| | 14.45% solids | | |
| Vacuum filter | | | Recover 3300 ml protein |
| Oxidise protein | To pH 11.3 | | |
| H₂O₂ (10%) | 300 | ml | |
| Diafilter (with 1 mM EDTA) | 33 | L | |
| Concentrate protein | 1600 | ml | |
| Protein concentration | 8.4% | | |

### Example 5c

| Wool (cut up) | 300 | g | 70 minutes ® 30° C. |
|---|---|---|---|
| Water | 4 | L | |
| Na₂S (as 100% Na₂S) | 16 | g | |
| NaOH (50%) | 50 | ml | |
| Centrifuge | | | 7000 rpm for 10 minutes |
| Solids recovered | 1253 g @ | | Total Wool digested 54% |
| | 10.94% solids | | |
| Vacuum filter | | | Recover 3100 ml protein |
| Oxidise protein | To pH 11.3 | | |
| H₂O₂ (10%) | 360 | ml | |
| Diafilter (with 1 mM EDTA) | 31 | L | |
| Concentrate protein | 1350 | ml | |
| Protein concentration | 5.16% | | |

### Example 5d

| Wool (cut up) | 300 | g | 70 minutes @ 45° C. |
|---|---|---|---|
| Water | 4 | L | |
| Na₂S (as 100% Na₂S) | 16 | g | |
| NaOH (50%) | 50 | ml | |
| Centrifuge | | | 7000 rpm for 10 minutes |
| Solids recovered | 873.5 g @ | | Total Wool digested 67% |
| | 11.4% solids | | |
| Vacuum filter | | | Recover 3200 ml protein |
| Oxidise protein | To pH 11.3 | | |
| H₂O₂ (10%) | 340 | ml | |
| Diafilter (with 1 mM EDTA) | 32 | L | |
| Concentrate protein | 1400 | ml | |
| Protein concentration | 4.74% | | |

For both wool and feather, the results indicate that at the higher temperature of 45° C., there is an increase in material digested by up to 14%. Tables 1 and 2 detail the results. If temperatures were further increased, it is expected that the volume of material digested would also increase. Energy consumption when scaled to industrial production was taken into account in selecting 45° C. as the temperature. It is noted that wool requires a higher solid:liquid ratio for digestion (reduction). Avoidance of hydrolysis of the protein, and achieving the appropriate molecular weight of proteins, was also taken into account in optimising the time and temperature conditions.

TABLE 1
Wool digested (300 g, 70 min) at various
temperatures (oxidised to pH 11.3)

| | |
|---|---|
| 30° C. | 45° C. |

| | | | | |
|---|---|---|---|---|
| Vol Recovered | 3100 | ml | 3200 | ml |
| H₂O₂ Required (10%) | 360 | ml | 340 | ml |
| % solid digested | 54% | | 67% | |

TABLE 2
Feather digested (500 g, 45 min) at various
temperatures (oxidised to pH 11.3)

| | |
|---|---|
| 30° C. | 45° C. |

| | | | | |
|---|---|---|---|---|
| Vol Recovered | 2800 | ml | 3300 | ml |
| H₂O₂ Required (10%) | 285 | ml | 300 | ml |
| % solid digested | 45% | | 59% | |

Further tests were conducted to assess appropriate conditions for oxidising protein to quantitate conversion of thiol groups to cysteic acid residues. When the protein solution is oxidised, significantly more hydrogen peroxide is required to oxidise to lower pH values. Tables 3 and 4 which contain the results of these tests demonstrate this. The amino acid analysis results reported in Example 7 also indicate that oxidising to lower than pH 11.3 is not required.

TABLE 3
Wool digested (300 g, 70 min) and oxidised
to various pH's (at 30° C.)

| | pH 11.3 | | pH 10.8 | | pH 10.0 | |
|---|---|---|---|---|---|---|
| Vol Recovered | 2300 | ml | 2000 | ml | 2000 | ml |
| H₂O₂ Required (10%) | 450 | ml | 200 | ml | 500 | ml |
| % solid digested | 56% | | 53% | | 53% | |
| Vol Conc. | 1600 | ml | 1150 | ml | 1200 | ml |
| Protein Conc | 4.38% | | 4.18% | | 3.92% | |
| Protein Produced (g) | 70.08 | | 48.07 | | 47.04 | |

TABLE 4
Feather digested (500 g, 45 min) and oxidised
to various pH's (at 30° C.)

| | pH 11.3 | | pH 10.8 | | pH 10.0 | |
|---|---|---|---|---|---|---|
| Vol Recovered | 2000 | ml | 2000 | ml | 2000 | ml |
| H₂O₂ Required (10%) | 150 | ml | 200 | ml | 500 | ml |
| % solid digested | 35% | | — | | — | |
| Vol Conc. | 1250 | ml | 1250 | ml | 950 | ml |
| Protein Conc | 5.87% | | 5.33% | | 5.98% | |
| Protein Produced (g) | 73.375 | | 66.625 | | 56.81 | |

### Example 6

Protein samples isolated from each of Examples 2 to 5 above were analysed by SDS-PAGE electrophoresis to assess the molecular weight of the extracted protein. The results of various samples are set out below.

### Procedure:

A Ready Gel Mini Gel System was used. This technique separates protein mixtures by molecular weight. When compared with known standards it is possible to estimate the molecular weight of an unknown protein sample. (For further information see the information on SDS-PAGE gels in the Bio-Rad 2004/2005 Life Science Research Products catalogue).

The protein samples to be analysed were prepared in a reducing denaturing sample buffer containing 2-mercaptoethanol. Two types of Bio-Rad precast gels were used, Tris-HCl and Tris-Tricine. These accurately establish the molecular weight of the wool and feather protein, respectively. The Tris-HCl 15% precast gel was run with Tris/glycine/SDS buffer. The Tris-Tricine/peptide ready gels are specifically for separating proteins less than 10 kDa. A Tris-Tricine 10-20% linear gradient gel was run with Tris-Tricine/SDS buffer. The two gels have good resolving capacities for the wool proteins and feather proteins. Illustrations of the resolving capacities can be obtained from Bio-Rad.

Bio-Rad Polypeptide SDS-PAGE standards are made from polypeptides. They have low molecular weight markers at 1.4, 3.4, 6.5, 14.4, 16.9 and 26.6 kDa.

Precision Plus Protein Standards are recombinant proteins and contain 10 protein markers at molecular weights 10, 15, 20, 25, 37, 50, 75, 100, 150, 250 kDa including 3 reference bands (25, 50 and 75 kDa).

The gels were stained with Coomassie Brilliant Blue R-250 for 30 minutes, followed by destaining to highlight the molecular weight bands.

### Results:

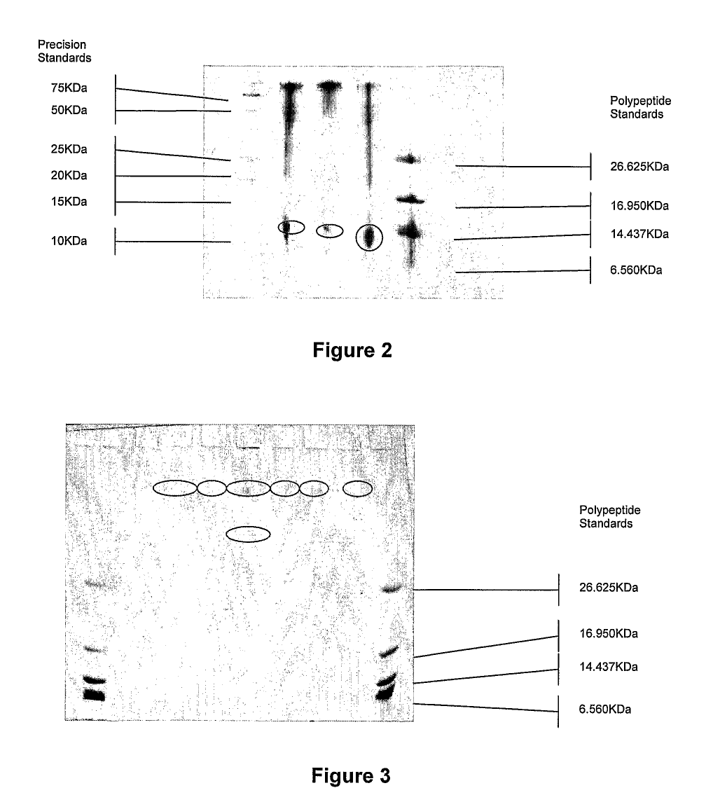

The Tris-Tricine gels produced clear indications regarding the molecular weight of the feather protein. FIG. 5 shows three oxidised feather protein samples that have molecular weights above 10 kDa in Tris-Tricine 10-20% gradient gel. The five lanes in FIG. 2, from left to right are as follows: Lane 1—Precision Standard, Lane 2—Oxidised Feather@30° C., Lane 3—Oxidised Feather@45° C., Lane 4—Reduced Feather@30° C., Lane 5—Polypeptide Standard.

Standard markers at 6.56 kDa and 10 kDa indicate that there is no protein with a molecular weight below 10 kDa. The gel does indicate some lightweight bands at about 50 kDa. The gel was run with both precision and polypeptide standards which behave a little differently and therefore do not align precisely. This does not detract from the clear band for the tested samples being above 10 kDA.

In FIG. 3 the results of the molecular weight analysis for keratinous protein extracted from wool in Tris-HCl 15% gel are presented. There are 10 lanes, with lanes 1 and 3 empty. The lanes from left to right are as follows: Lane 2—Std, Lane 4—pH 11.3 (1:5), Lane 5—pH 11.3 (1:10), Lane 6—pH 10.8 (1:5), Lane 7 pH 10.8 (1:10), Lane 8—pH 10.1 (1:5), Lane 9—pH (1:10) Lane 10—Standard.

FIG. 3 indicates that there are very few low molecular weight proteins in wool. Literature indicates that the majority of wool proteins are in the 45-60 kDa range. The results also show that the digestion and oxidation techniques used do not produce significant amounts of protein below 10 kDa.

### Example 7

Tests were conducted to optimise the pH of the oxidation step of the process outlined in Example 4, and the amino acid levels of the extracted proteins were quantitatively analysed to confirm the results.

### Procedure:

A Waters Alliance HPLC system was used and controlled via Waters proprietary Enpower software. A Waters cation exchange column (Wat080002) was employed with two eluting buffers:

Buffer A pH 2.96 [0.2M] Na⁺

Buffer B pH 6.50 [1.2M] Na⁺

The elution temperature was 65° C.

Ninhydrin post column reaction with reaction temp of 125° C. was used to develop a chromophore detected by a Waters 2487 UV/Vis detector. Primary amino acids were detected at 570 nm and secondary amino acids at 440 nm. Enpower software was used for data collection and calculation.

### Results:

All keratins have a large number of disulfide bonds (—S—S—) which must be broken to solubilise the protein. Feathers have a distinct amino acid composition with a high proportion of glycine, serine and proline. They also have a low level of histidine, lysine and methionine. In the alkaline digestion process, the disulfide bonds in the cystine residues are reduced to cysteine. The oxidation process prevents the cysteine residues from reforming these disulfide bonds.

The digestion conditions were compared for oxidising protein to quantitate conversion of cystine thiol —SH groups to cysteic acid residues. Raw feather contains 100% cystine disulfide bonds. Digestion and subsequent oxidation to various pH levels changes the ratio of the thiol —SH groups to cysteic acid residues. The data in Table 5 indicates that pH 11.3 is the optimum pH to obtain maximum conversion. At lower pH's, the ratio is closer to 50:50 and indicates reformation of disulfide linkages. A small amount of this is generally acceptable, so lower pH's may be used in the process—although not less than 10.0. The different temperatures of 30° C. and 45° C. have not affected the conversion.

TABLE 5

| | | Feather oxid | Feather oxid | Feather oxid | Feather oxid |
|---|---|---|---|---|---|
| | | pH 10 @ | pH 10.8 @ | pH 11.3 @ | pH 11.3 @ |
| | Raw Feather | 30° C. | 30° C. | 30° C. | 45° C. |
| Amino Acid | % total | % total | % total | % total | % total |
| 1/2cys (—SH) | 7.171 | 4.947 | 4.266 | 0.843 | 0.991 |
| cysA (—SO₃⁻) | 0.000 | 3.071 | 3.451 | 5.322 | 4.072 |

As expected, the results for wool are similar and follow the same trend. These are detailed in Table 6.

TABLE 6

| | | Wool oxid | Wool oxid | Wool oxid | Wool oxid |
|---|---|---|---|---|---|
| | | pH 10.1 @ | pH 10.8 @ | pH 11.3 @ | pH 11.3 @ |
| | Raw Wool | 30° C. | 30° C. | 30° C. | 45° C. |
| Amino Acid | % total | % total | % total | % total | % total |
| 1/2cys (—SH) | 10.602 | 2.367 | 5.492 | 0.448 | 1.711 |
| cysA (—SO₃⁻) | 0.199 | 5.690 | 2.646 | 6.751 | 5.524 |

It will be understood to persons skilled in the art of the invention that many modifications may be made to the preferred embodiments described in the examples without departing from the spirit and scope of the invention.

1. A process for separating keratinous proteins from a keratin-containing material, the process comprising the steps of:
i. subjecting the keratin-containing material to reduction for a time and at a temperature in a liquid medium to solubilise the keratinous proteins, to yield a solution of keratinous proteins and undissolved solids,
ii. subjecting the solution of keratinous proteins to peroxide oxidation, without any intervening keratin precipitation step; and
iii. separating the solution of keratinous proteins from the undissolved solids prior to, at the same time as, or following the oxidation step.

2. The process of claim 1, wherein the solution of keratinous proteins comprises unhydrolysed keratinous proteins.

3. The process of claim 1, wherein the solution of keratinous proteins comprises keratinous proteins having a molecular weight above 10 kDa.

4. The process of claim 1, wherein the keratin-containing material is feather and/or wool.

5. The process of claim 1, wherein the keratin-containing material is washed or fragmented prior to reduction.

6. (canceled)

7. The process of claim 1, wherein the time between 30 minutes and 1.5 hours, and the temperature is between 25° C. and 50° C., based on atmospheric pressure.

8. The process of claim 1, wherein the liquid medium comprises alkali metal sulfide.

9. The process of claim 8, wherein the liquid medium comprises alkali metal sulfide in an amount between 5 and 1000 grams per 100 litres.

10. The process of claims 1, wherein the reduction is conducted in the presence of an alkali.

11. The process of claim 10, wherein the reduction conducted in the presence of alkali adjusts the pH to a value in the range of 12.0-13.5.

12. (canceled)

13. The process of claim 1, wherein the separation step is conducted within a period not greater than 4 hours after the reduction step.

14. The process of claim 1, wherein the solution of keratinous proteins is separated from the undissolved solids prior to the peroxide oxidation.

15. The process of claim 14, wherein the solution of keratinous proteins is separated from the undissolved solids prior to peroxide oxidation, and without any intervening treatment steps.

16. (canceled)

17. The process of claim 1, wherein the peroxide oxidation step commences not more than 6 hours after the commencement of the reduction step.

18. The process of claim 1, wherein cysteine residues of the keratinous proteins are fully oxidized after the peroxide oxidation step.

19. The process of claim 1, wherein the peroxide is used in an amount to reduce the pH to a level not below 10.0.

20. (canceled)

21. The process of claim 1, wherein the solution of keratinous proteins is not reduced below pH 10, prior to completion of the peroxide oxidation step.

22. The process of claim 1, wherein the process comprises the further step of:
iv. neutralising the oxidised solution of keratinous proteins.

23. The process of claim 22, wherein the process comprises the further step of:
v. desalting the neutralised solution of keratinous proteins.

24. (canceled)

25. (canceled)

26. (canceled)

27. The process of claim 22 or claim 23, wherein the process comprises the further step of
modifying a side chain of a keratinous protein.

28. (canceled)

29. (canceled)

30. (canceled)

31. (canceled)
